SEQUENCE LISTING (5 sequences)

SEQ ID NO: 1 - DNA, 15 bp, Mus musculus
  misc_feature (7)..(9): n is a, c, g, or t
gggccannntgaccc

SEQ ID NO: 2 - DNA, 15 bp, Mus musculus
  misc_feature (7)..(9): n is a, c, g, or t
aggtcannntgacct

SEQ ID NO: 3 - DNA, 13 bp, Mus musculus
  misc_feature (6)..(8): n is a, c, g, or t
ggccannntgacc

SEQ ID NO: 4 - DNA, 15 bp, Mus musculus
gggccactctgaccc

SEQ ID NO: 5 - DNA, 15 bp, Mus musculus
gaaccactctacctc

(19) United States
(12) Patent Application Publication
(10) Pub. No.: US 2005/0271637 A1
(43) Pub. Date: Dec. 8, 2005

(54) BMP-2 estrogen responsive element and methods of using the same

(76) Inventors: Peter Van Nest Bodine, Havertown, PA (US); Dan Gazit, Jerusalem (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/642,503

(22) Filed: Aug. 18, 2003

Related U.S. Application Data
(60) Provisional application No. 60/404,024, filed on Aug. 16, 2002.

(51) Int. Cl. C12Q 1/68; C07H 21/04; A61K 48/00; C12N 5/08

(52) U.S. Cl. 424/93.21; 435/6; 435/69.1; 435/366; 530/399; 536/23.2

(57) ABSTRACT

The invention relates to an isolated nucleic acid corresponding to BMP-2 regulatory region, or a fragment thereof comprising an estrogen responsive element, vector comprising the same and cells, which comprises said vector. In another embodiment, the invention provides methods of identifying an estrogen agonist, antagonist and a therapeutic agent in another embodiment the invention provides methods of treating conditions which are associated with estrogen insufficiency or with lack of response to external estrogen or agonists thereof.

### CROSS REFERENCE TO RELATED APPLICATION

This application claims the priority of U.S. Provisional Application Ser. No. 60/404,024, filed on Aug. 16, 2002, which is incorporated herein by reference.

### BACKGROUND OF THE INVENTION

Throughout adult life, bone is continually undergoing remodeling through the interactive cycles of bone formation and resorption (bone turnover). Bone resorption typically is rapid, and is mediated by osteoclasts (bone resorbing cells), formed by mononuclear phagocytic precursor cells at bone remodeling sites. This process is followed by the appearance of osteoblasts (bone forming cells), which form bone slowly to replace the lost bone. The activities of the various cell types that participate in the remodeling process are controlled by interacting systemic (e.g., hormones, lymphokines, growth factors, vitamins) and local factors (e.g., cytokines, adhesion molecules, lymphokines and growth factors). The fact that completion of this process normally leads to balanced replacement and renewal of bone indicates that the molecular signals and events that influence bone remodeling are tightly controlled.

A number of bone growth disorders are known which cause an imbalance in the bone remodeling cycle. Chief among these are metabolic bone diseases, such as osteoporosis, osteoplasia (osteomalacia), chronic renal failure and hyperparathyroidism, which result in abnormal or excessive loss of bone mass (osteopenia). Other bone diseases, such as Paget's disease, also cause excessive loss of bone mass at localized sites.

Osteoporosis is a structural deterioration of the skeleton caused by loss of bone mass resulting from an imbalance in bone formation, bone resorption, or both, such that the resorption dominates the bone formation phase, thereby reducing the weight-bearing capacity of the affected bone. In a healthy adult, the rate at which bone is formed and resorbed is tightly coordinated so as to maintain the renewal of skeletal bone. However, in osteoporotic individuals an imbalance in these bone remodeling cycles develops which results in both loss of bone mass and in formation of microarchitectural defects in the continuity of the skeleton. Osteoporosis affects about 50% of women, and about 10% of men, over the age of 50 in the United States. In individuals with osteoporosis, increased loss of bone mass results in fragile bones and, as a result, increased risk of bone fractures. Other bone-resorption diseases, such as Paget's disease and metastatic bone cancer, present similar symptoms.

Bone morphogenetic proteins (BMPs) are members of the transforming growth factor β (TGF-β) superfamily and originally identified by their presence in bone-inductive extracts of demineralized bone (Wozney et al., 1988; Rosen et al., 1996). It has long been suspected that the primary target cells for BMP action is an early osteoblast progenitor or the mesenchymal stem cell (Oreffo et al., 1999). Recombinant human BMP-2, a member of the BMP family, induces cartilage and bone formation in vivo (Wozney et al 1988, Wang et al 1990, Gazit et al 1999) and osteogenic differentation of several mesenchymal cell types in vitro (Katagiri et al 1990; Theis et al 1992; Wang et al 1993; Yamaguchi et al 1996; Hanada et al 1997; Gazit et al 1999; Moutsatsos et al 2001; Turgeman et al 2001).

### SUMMARY OF THE INVENTION

In one embodiment, the invention provides an isolated nucleic acid comprising a nucleic acid sequence, which corresponds to a BMP-2 regulatory region, or a fragment thereof comprising an estrogen responsive element.

In another embodiment, the invention provides a vector comprising the isolated nucleic acid comprising a nucleic acid sequence which corresponds to a BMP-2 regulatory region, or a fragment thereof comprising an estrogen responsive element and is operably linked to a second nucleic acid.

In another embodiment, this invention provides a host cell comprising an isolated nucleic acid comprising a nucleic acid sequence which corresponds to a BMP-2 regulatory region, or a fragment thereof comprising an estrogen responsive element.

In another embodiment, this invention provides a method for the identification of a potential therapeutic agent for the prevention and/or treatment of osteoporosis, comprising: (a) introducing into a cell a vector comprising an isolated nucleic acid corresponding to BMP-2 regulatory region, or a fragment thereof that comprises an estrogen responsive element and is operably linked to a reporter gene, (b) contacting the cell with a candidate agent; and (c) monitoring the expression of the protein encoded by the reporter gene, wherein induced expression of the protein indicates that the candidate agent is a potential therapeutic agent.

In another embodiment, this invention provides a method of regulating expression of BMP-2 in a subject comprising the steps of administering a vector comprising an isolated nucleic acid corresponding to BMP-2 regulatory region, or a fragment thereof that comprises an estrogen responsive element and is operably linked to a second nucleic acid; and administering to the subject an effective amount of estrogen or estrogen agonist; thereby regulating expression of BMP-2 in the subject.

In another embodiment, this invention provides a method of regulating expression of BMP-2 in a subject comprising the steps of: administering to the subject an effective amount of a cell comprising an isolated nucleic acid corresponding to BMP-2 regulatory region, or a fragment thereof that comprises an estrogen responsive element and is operably linked to a nucleic acid that encodes the BMP-2; and administering to the subject in need an effective amount of estrogen or estrogen agonist; thereby regulating expression of BMP-2 in the subject.

In another embodiment, this invention provides a method of increasing responsiveness of a cell to estrogen or estrogen agonist comprising the step of administering a vector comprising an isolated nucleic acid corresponding to BMP-2 regulatory region, or a fragment thereof comprising an estrogen responsive element and is operably linked to a second nucleic acid; thereby increasing the responsiveness of the cell to estrogen.

In another embodiment, this invention provides a method of enhancing repair of a bone in the body in a subject in need comprising the steps of: administering an isolated nucleic acid corresponding to BMP-2 regulatory region, or a fragment thereof comprising an estrogen responsive element and is operably linked to a second nucleic acid of interest; and administering to the subject in need an effective amount of estrogen or estrogen agonist; thereby enhancing repair of the bone in the body of the subject in need.

In another embodiment, this invention provides a method of enhancing repair of a bone comprising the steps of: administering to a subject an effective amount of a cell a host cell comprising an isolated nucleic acid corresponding to BMP-2 regulatory region, or a fragment thereof comprising an estrogen responsive element and is operably linked to a second nucleic acid; and administering to the subject in need an effective amount of estrogen or estrogen agonist; thereby enhancing repair of the bone in the subject.

In another embodiment this invention provides a method for maintaining or increasing bone volume, bone quality, or bone strength in a subject in need afflicted with osteoporosis caused by or accompanied by a decrease in estrogen comprising the steps of: administering a vector comprising an isolated nucleic acid corresponding to BMP-2 regulatory region, or a fragment thereof comprising an estrogen responsive element and is operably linked to a second nucleic acid; and administering to the subject in need an effective amount of estrogen or estrogen agonist; thereby maintaining or increasing bone volume, bone quality, or bone strength in the subject in need.

In another embodiment, this invention provides a method for maintaining or increasing bone volume, bone quality, or bone strength in a subject in need afflicted with osteoporosis caused by or accompanied by a decrease in estrogen comprising the steps of: administering to a subject an effective amount of a cell a host cell comprising an isolated nucleic acid corresponding to BMP-2 regulatory region, or a fragment thereof comprising an estrogen responsive element and is operably linked to a second nucleic acid; and administering to the subject in need an effective amount of estrogen or estrogen agonist; thereby maintaining or increasing bone volume, bone quality, or bone strength in the subject in need.

In another embodiment, this invention provides a method of enhancing repair of a bone in the body in a subject in need comprising the steps of: obtaining a cell from of the subject; transfecting the cell with a vector comprising an isolated nucleic acid corresponding to BMP-2 regulatory region, or a fragment thereof comprising an estrogen responsive element and is operably linked to a second nucleic acid; administering the engineered cell to the subject; and administering to the subject in need an effective amount of estrogen or estrogen agonist; thereby enhancing repair of a bone in the body in the subject in need.

In another embodiment, this invention provides a method for maintaining or increasing bone volume, bone quality, or bone strength in a subject in need afflicted with osteoporosis caused by or accompanied by a decrease in estrogen comprising the steps of: obtaining a cell from of the subject; transfecting the cell with a vector comprising an isolated nucleic acid corresponding to BMP-2 regulatory region, or a fragment thereof comprising an estrogen responsive element and is operably linked to a second nucleic acid, administering the engineered cell to the subject; and administering to the subject in need an effective amount of estrogen or estrogen agonist; thereby maintaining or increasing bone volume, bone quality, or bone strength in a subject in need.

In another embodiment, this invention provides a method for the production of transplantable bone matrix, the method comprising the steps of: obtaining a cell; transfecting the cell with a vector comprising an isolated nucleic acid corresponding to BMP-2 regulatory region, or a fragment thereof that comprises an estrogen responsive element and is operably linked to a second nucleic acid; and culturing the cell with the cell-associated matrix for a time effective for allowing formation of a transplantable bone matrix.

In another embodiment, this invention provides a method of stimulating osteoblast differentiation comprising the steps of: administering a vector comprising an isolated nucleic acid corresponding to BMP-2 regulatory region, or a fragment thereof comprising an estrogen responsive element and is operably linked to a second nucleic acid; and administering an effective amount of estrogen or estrogen agonist; thereby stimulating osteoblast differentiation.

In another embodiment, this invention provides a method of treating a bone disease in a subject comprising the steps of: administering a vector comprising an isolated nucleic acid corresponding to BMP-2 regulatory region, or a fragment thereof comprising an estrogen responsive element and is operably linked to a second nucleic acid; and administering to the subject an effective amount of estrogen or estrogen agonist; thereby treating a bone disease in the subject.

In another embodiment, this invention provides a method of treating a bone disease in a subject comprising the steps of: administering to the subject an effective amount of a cell a host cell comprising an isolated nucleic acid corresponding to BMP-2 regulatory region, or a fragment thereof comprising an estrogen responsive element and is operably linked to a gene; and administering to the subject in need an effective amount of estrogen or estrogen agonist; thereby treating a bone disease in the subject.

In another embodiment this invention provides a method for identifying a compound in a sample as an estrogenic agonist comprising: (a) providing a cell line expressing receptors for human estrogen, which cell line has been stably transfected by a vector comprising a reporter gene operatively linked to a an isolated nucleic acid corresponding to BMP-2 regulatory region, or a fragment thereof which an estrogen responsive element which estrogen responsive element is capable of controlling expression of the reporter gene in response to estrogen; (b) contacting the transfected cell line with a sample suspected to contain a human estrogen agonist, under conditions in which human estrogen would cause increased expression of the reporter gene; and (c) measuring the level of expression of the reporter gene, whereby a human estrogen agonist in the sample is identified by measurement of an increased level of expression of the reporter gene, compared to the level produced by a buffer control.

In another embodiment this invention provides a method for identifying a compound in a sample as a human estrogen antagonist comprising: (a) providing a cell line expressing receptors for human estrogen, which cell line has been stably transfected by a vector comprising a reporter gene operatively linked to a an isolated nucleic acid corresponding to BMP-2 regulatory region, or a fragment thereof comprising an estrogen responsive element, which estorgen responsive element is capable of controlling expression of the reporter gene in response to estrogen; (b) contacting the transfected cell line with a sample suspected to contain a human estrogen antagonist, to which has been added an amount of human estrogen that, absent such antagonist, would produce a measurable increase in expression of the reporter gene; and (c) measuring the level of expression of the reporter gene, whereby a human estrogen antagonist in the sample is identified by measurement of a decreased level of expression of the reporter gene, compared to the level produced by the human estrogen in the absence of such antagonist.

### BRIEF DESCRIPTION OF THE DRAWINGS

FIG. 1. E2 regulates mouse BMP-2 mRNA expression in MSCs obtained from OVX mice demonstrated by real-time RT-PCR. After 24 hr treatment with 100 nM E2, mouse BMP-2 mRNA levels were significantly increased from 570±81 copies to 1337±177 copies (p<0.05, ANOVA) in 2 μg of total RNA

FIG. 2. E2 directly regulates BMP-2 mRNA expression in MSCs obtained from ovariectomized mice. Five μM cycloheximide did not block the up-regulation of BMP-2 by estradiol (E2) treatment for 4 hr (A), although the same concentration of cycloheximide caused superinduction of c-myc (B).

FIG. 3. E2, but not selective estrogen receptor modulators, regulates BMP-2 mRNA expression via the estrogen receptor (ER) in MSCs obtained from overectomized mice. (A) ICI (10 μM) blocked the up-regulation of BMP-2 mRNA expression in MSCs by treatment with E2 (10⁻⁷ M) for 24 hr as shown by semi-quantitative RT-PCR. (B) BMP-2 mRNA expression was up-regulated in MSCs by E2 (10-7 M) treatment for 24 hr, but not by tamoxifen (10-6 M) or raloxifene (10-7 M).

FIG. 4. Wild-type mouse C3H10T1/2 cells do not express functional ERs and require transfection of either ERα or ERβ. (A) RNA was isolated from either wild-type (WT) or stable C3H10T1/2 cell lines that over-expressed either human ERα or human ERβ and RT-PCR is performed for the ERs or GADPDH. Lanes: M, 1 kb molecular weight ladder; 1, WT cells analyzed for ERα; 2, ERβ cells analyzed for ERβ; 3, ERβ cDNA control; 4, WT cells analyzed for GAPDH; 5, Erβ cells analyzed for GAPDH; 6, WT cells analyzed for ERα; 7, ERα cells analyzed for ERα; 8, ERα cDNA control; 9, WT cells analyzed for GAPDH; 10, Erα cells analyzed for GAPDH. (B) Either wild-type (WT) or stable C3H10T1/2 cell lines that over-expressed either human ERα or human ERβ were transiently transfected with ERE-tk-luciferase plasmid, treated with 10 nM E2 for 24 hr, and assayed for luciferase activity by a luminometer.

FIG. 5. E2 stimulates mouse BMP-2 promoter activity via ERα and ERβ. E2 regulated dose-dependently full-length mouse BMP-2 promoter (−2712) (B) and the classical estrogen responsive element (ERE) (C) activity via ERs. Five μg of BMP-2 promoter-luciferase plasmid (BMP-2 full-length promoter linked to luciferase in the pGL3 vector) or ERE-tk-luciferase plasmid were transiently co-transfected into mouse C3H10T1/2 cells with 2 μg each of either human ERα or human ERβ expression vectors. The cells were then treated with different doses of E2 for 24 hr, and luciferase activity was assayed by luminometer.

FIG. 6. ICI-182, 780 dose-dependently inhibits the stimulation of E2 on mouse BMP-2 promoter activity via ERα and ERβ. Mouse C3H10T1/2 cells were transfected with mouse BMP-2 promoter-Luciferase vectors (−2712) and ERα (A) or ERβ (B) expression vectors as described in FIG. 5.

FIG. 7. The location of the ER regulation site in the mouse BMP-2 promoter. Specific deletions of the mouse BMP-2 promoter were obtained by digestion with restriction enzymes (−838 and −150) from the full-length promoter (−2712). The promoter fragments were then subcloned as PCR products into the pGL3-basic vector (−448 to +23 and −400 to +23). Mutation of the wild-type BMP-2 promoter variant ERE (Δvariant ERE: 5′-GAACCActcTACCTC-3′) in the full-length promoter-luciferase plasmid was accomplished as described in the Materials and Methods.

FIG. 8. The effects of E2, SERMs and genistein on mouse BMP-2 promoter activity via ERα and/or ERβ. BMP-2 promoter-Luciferase vectors (−2712) were transiently transfected into C3H10T1/2 cells with hERα or hERβ expression vectors as described in FIG. 5. The cells were treated with 10 nM E2, 10 uM tamoxifen, 100 nM raloxifene, 100 nM ICI-182, 780, or 100 nM genistein.

FIG. 9. Models of ER action at the variant estrogen responsive element of the mouse BMP-2 promoter.

### DETAILED EMBODIMENTS OF THE INVENTION

The invention is directed to an isolated nucleic acid comprising a nucleic acid sequence which corresponds to a BMP-2 regulatory region comprising an estrogen responsive element, vectors comprising the same and cells which comprises said vector. In another embodiment, the invention provides methods of identifying an estrogen agonist, antagonist and a therapeutic agent; in another embodiment the invention provides methods of treating conditions which are associated with estrogen insufficiency or with lack of response to external estrogen or agonists thereof.

In one embodiment, the invention provides an isolated nucleic acid corresponding to BMP-2 regulatory region, or a fragment thereof comprising an estrogen responsive element.

In one embodiment, an “estrogen responsive element” is a nucleic acid sequence, which when operatively associated with a promoter, renders the promoter inducible by estrogen. As a result of such association, cells stably transformed by a vector comprising a reporter gene operatively linked to a nucleic acid which corresponds to BMP-2 regulatory region, or a fragment thereof that comprises an estrogen responsive element wherein increased levels of the reporter gene product are produced in the presence of estrogen or estrogen agonists.

In one embodiment, the invention provides a nucleic acid, which is at least 95% homologous to a BMP-2 regulatory region, or a fragment thereof that comprises an estrogen responsive element. In another embodiment, the invention provides a nucleic acid, which is at least 90% homologous to a BMP-2 regulatory region, or a fragment thereof that comprises an estrogen responsive element. In another embodiment, the invention provides a nucleic acid, which is at least 85% homologous to a BMP-2 regulatory region, or a fragment thereof that comprises an estrogen responsive element. In another embodiment, the invention provides a nucleic acid, which is at least 80% homologous to a BMP-2 regulatory region, or a fragment thereof that comprises an estrogen responsive element. In another embodiment, the invention provides a nucleic acid, which is at least 77% homologous to a BMP-2 regulatory region, or a fragment thereof that comprises an estrogen responsive element. In another embodiment, the invention provides a nucleic acid sequence, which is at least 70% homologous to BMP-2 regulatory region, or a fragment thereof that comprises an estrogen responsive element. In another embodiment, the invention provides a nucleic acid which is between 70% and 100% homologous to a BMP-2 regulatory region, or a fragment thereof that comprises an estrogen responsive element.

In one embodiment, a “BMP-2 regulatory region, or a fragment thereof that contains an estrogen responsive element” is a BMP-2 gene that is inducible by estrogen or estrogen agonist. As a result of this induction, cells stably transformed by a vector comprising a reporter gene operatively linked to an isolated nucleic acid corresponding to BMP-2 regulatory region, or a fragment thereof that comprises an estrogen responsive element produce increased levels of the reporter gene product (for example, without limitation of BMP-2) in the presence of human estrogen.

In another embodiment, the isolated nucleic acid corresponding to BMP-2 regulatory region, or a fragment thereof that comprises an estrogen responsive element has the nucleic acid sequence of SEQ ID No. 1. In the mouse BMP-2 promoter sequence, applicants found a variant non-palindromic ERE (5′-GGGCCAnnnTGACCC-3′) (SEQ ID NO: 1) located at −415 to −402. The mouse BMP-2 variant ERE has a 3 bp change from the classical vitellogenin A2 ERE (5′-AGGTCAnnnTGACCT-3′) (SEQ ID NO: 2) over a 15 bp sequence. However, over the core 13 bp consensus ERE sequence (5′-GGCCAnnnTGACC-3′) (SEQ ID NO: 3), only one base pair is altered. As provided herein, by comparing the activity of different deletions of the mouse BMP-2 promoter and mutation of the BMP-2 variant ERE, it was shown that the regulation of the promoter by ERα and ERβ is via this variant ERE binding site and not via the AP-1 or Sp1 sites.

DNA that encodes the BMP-2 regulatory region or fragment thereof that contains the estrogen responsive element of the invention may be obtained, in view of the instant disclosure, by chemical synthesis, by in vitro amplification [including but not limited to the polymerase chain reaction (PCR)], or by combinations of these procedures from naturally-occurring sources, such as cultures of mammalian cells, genomic DNA from such cells or libraries of such DNA.

The isolated nucleic acid corresponding to BMP-2 regulatory region, or a fragment thereof that comprises an estrogen responsive element of the invention may be operably linked to reporter genes and used to either transiently or stably transform appropriate host cells through the use of appropriate vectors, constructs and means well known in the art, such as DNA mediated gene transfer means including but not limited to transfection, electroporation and virally-mediated infection. If viruses are used, the virus used may be in one embodiment adenovirus.

In another embodiment, the vector is a DNA molecule comprising the regulatory elements necessary for transcription of a gene in a host cell. Typically the gene is placed under the control of certain regulatory elements including constitutive or inducible promoters, tissue-specific regulatory elements, and enhancer elements. Such a gene is said to be “operably linked to” the regulatory elements when the regulating element controls the expression of the gene. Expression vectors typically include eukaryotic and/or bacterial selectable markers that allow for selection of cells containing the expression vector.

In another embodiment, the invention provides a vector comprising an isolated nucleic acid corresponding to BMP-2 regulatory region, or a fragment thereof that comprises an estrogen responsive element and is operably linked to a second nucleic acid.

Insertion of the promoters and reporter genes into a vector is easily accomplished when the termini of both the DNAs containing such elements and the vector comprise compatible restriction sites.

Alternatively, any desired site may be produced by ligating nucleotide sequences (linkers) onto the termini. Such linkers may comprise specific oligonucleotide sequences that define desired restriction sites. The cleaved vector and the DNA fragments may also be modified if required by homopolymeric tailing.

The responsive elements can be inserted into many mammalian reporter gene-containing vectors, including but not limited to plasmids pSV2Apap, pMAMneo-CAT, pMAMneo-LUC, pSVOCAT, pBCO, pBLCAT2, pBLCAT3, pON1, pCH110, p.O slashed.GH, pIL-4 RE-SV40-LacZ, pSP72 and various plasmids described by De Wet et al., where a desired vector contains a different promoter, such promoter can be excised using standard methods and replaced by a BMP-2 regulatory region or fragment thereof that contains an estrogen responsive element. Alternatively, the estrogen responsive element can be placed in association with another promoter to render it inducible by estrogen.

The above-mentioned recombinant vectors can be used to stably transform any mammalian cell that is capable of responding to estrogen or agonist thereof, i.e which includes receptors which responds to estrogen or estrogen agonist. To date, there are two known types of estrogen receptors, which are estrogen receptor α and estrogen receptor β.

In another embodiment, the invention provides a host cell comprising an isolated nucleic acid corresponding to BMP-2 regulatory region, or a fragment thereof that comprises an estrogen responsive element and is operably linked to a second nucleic acid.

In another embodiment, the cell of the invention may be modified to provide truncated or chimeric estrogen receptors, or natural estrogen receptors as described in Berry, et al., E. M. B. O. J., 9:2811-2818 (1990). These modifications may result in increased estrogen affinity and increased sensitivity and will increase the efficacy of the therapy.

In another embodiment, the cell of the invention may be an osteoblast, a mesenchymal stem cell a progenitor cell or a cell, which may be differentiated into an osteoblast.

In one embodiment, “a second nucleic acid” is any nucleic acid (gene), which is associated with conditions of estrogen insufficiency or with lack of responsiveness to estrogen by the subject. Nucleic acid of particular interest to be expressed in cells of a subject for treatment of genetic or acquired diseases include those encoding osteogenic factors or genes which associated other actions of estrogen such as those associated with cognitive functions, neuroprotection, enhancement of nerves regeneration and stimulation of neurite growth. In another embodiment the genes are associated with cancer, angiogenesis, stroke and cardiovacular diseases.

In another embodiment, the estrogen responsive element of the invention can be used to treat various bone diseases or conditions, which are associated with estrogen deficiency, or lack of response to estrogen. The treatment will result in higher expression of the products encode by the second nucleic acid.

In another embodiment the a second nucleic acid may be genes which encodes osteogenic factors such as OP-1, OP-2, BMP-5, BMP-6, BMP-2, BMP-3, BMP-4, BMP-9, DPP, Vg-1, 60A, Vgr-1.

In another embodiment, the expression of the product of the genes of interest will be increased by at least 1.5 fold. In another embodiment, the expression of the product of the BMP-2 will be increased by 1.5 fold to 30 fold.

Although cells used in the present invention could in principle be transiently transformed, stably-transformed cells are preferred. Stable transformation of a human cell line can be accomplished by using standard methods to co-transfect the cells with one of the above-mentioned recombinant vectors and with a second vector (such as pSV2neo or pRSVneo), which confers resistance to a selection agent such as an antibiotic. Alternatively, transformation can be carried out with a single vector containing both the promoter/reporter gene construct and the selection marker gene.

Quantitative real-time RT-PCR results indicted that E2 increases BMP-2 gene expression after 24 hr of treatment in mouse bone marrow-derived MSCs. Cotreatment with cycloheximide, an inhibitor of protein synthesis, did not block the up-regulation of BMP-2 mRNA by E2 treatment. However, the same concentration of the inhibitor caused a super-induction of c-myc mRNA levels implying that it blocked protein synthesis (Hauguel-de Mouzon and Kahn, 1991). Thus, these results indicate that E2 directly regulates BMP-2 mRNA levels. In addition, SERMs such as tamoxifen, raloxifene and ICI failed to activate mouse BMP-2 gene expression, while ICI inhibited E2 stimulation of gene expression. These results indicate that the increase in BMP-2 mRNA by E2 is ER dependent.

To determine the mechanism by which E2 transciptionally activates the mouse BMP-2 promoter, a model system was developed by transiently transfecting promoter-luciferase reporter gene constructs into pluripotent mouse mesenchymal C3H10T1/2 cells. Since C3H10T1/2 cells do not express ERs, they were co-transfected with expression vectors encoding either human ERα and/or ERβ (An et al. 1999). E2 dose-dependently induced mouse BMP-2 promoter activity in cells co-transfected with either ERα or ERβ. At a dose of 10 nM E2, ERα induced mouse BMP-2 promoter luciferase activity by 9.0-fold, while a 3.3-fold increase was observed in cells co-transfected with ERβ. ICI blocked the activation of mouse BMP-2 promoter activity by E2 via both ERα and ERβ, indicating that promoter activation is ER dependent. This result confirmed the RT-PCR results of BMP-2 mRNA expression in mouse bone marrow MSCs.

In another embodiment, the cell of the invention may be modified to provide truncated or chimeric estrogen receptors as described in Berry et al., E. M. B. O. J., 9:2811-2818 (1990). These modifications may result in increased estrogen affinity and increased sensitivity of the assay and when the cell is used for therapeutic purposes it will increase the efficacy of the therapy.

In another embodiment, this invention provides a method for the identification of a potential therapeutic agent for the prevention and/or treatment of osteoporosis, comprising: (a) introducing into a cell a vector comprising an isolated nucleic acid corresponding to BMP-2 regulatory region, or a fragment thereof comprising an estrogen responsive element and is operably linked to a reporter gene, (b) contacting the cell with a candidate agent; and (c) monitoring the expression of the protein encoded by the reporter gene, wherein induced expression of the protein indicates that the candidate agent is a potential therapeutic agent.

In another embodiment, this invention provides a method for identifying a compound in a sample as an estrogenic agonist comprising: (a) providing a cell line expressing receptors for human estrogen, which cell line has been stably transfected by a vector comprising a reporter gene operatively linked to an isolated nucleic acid corresponding to BMP-2 regulatory region, or a fragment thereof that comprises an estrogen responsive element which estrogen responsive element is capable of controlling expression of the reporter gene in response to estrogen; (b) contacting the transfected cell line with a sample suspected to contain a human estrogen agonist, under conditions in which human estrogen would cause increased expression of the reporter gene; and (c) measuring the level of expression of the reporter gene, whereby a human estrogen agonist in the sample is identified by measurement of an increased level of expression of the reporter gene, compared to the level produced by a buffer control.

In another embodiment, this invention provides a method for identifying a compound in a sample as a human estrogen antagonist comprising: (a) providing a cell line expressing receptors for human estrogen, which cell line has been stably transfected by a vector comprising a reporter gene operatively linked an isolated nucleic acid corresponding to BMP-2 regulatory region, or a fragment thereof comprising an estrogen responsive element, which is capable of controlling expression of the reporter gene in response to estrogen; (b) contacting the transfected cell line with a sample suspected to contain a human estrogen antagonist, to which has been added an amount of estrogen that, absent such antagonist, would produce a measurable increase in expression of the reporter gene; and (c) measuring the level of expression of the reporter gene, whereby a human estrogen antagonist in the sample is identified by measurement of a decreased level of expression of the reporter gene, compared to the level produced by the human estrogen in the absence of such antagonist.

In one embodiment, a “reporter gene” is a coding unit whose product is easily assayed (such as, without limitation, luciferase or chloramphenicol transacetylase). A reporter gene can be either a DNA molecule isolated from genomic DNA, which may or may not contain introns, or a complementary DNA (cDNA) prepared using messenger RNA s a template. In either case, the DNA encodes an expression product that is readily measurable, e.g., by biological activity assay, enzyme-linked immunosorbent assay (ELISA) or radioimmunoassay (RIA). Expression products of the reporter genes can be measured using standard methods. Various types of immunoassays such as competitive immunoassays, direct immunoassays and indirect immunoassays may be used.

Such immunoassays involve the formation of immune complexes containing the reporter gene product and a measurable label. In one embodiment, “label” includes moieties that can be detected directly, such as fluorochromes and radiolabels, and moieties such as enzymes that must be reacted or derivatized for detection.

The particular label used will depend upon the type of immunoassay used. Examples of labels that can be used include, e.g., radiolabels such as ³²P, ¹²⁵I, ³H and ¹⁴C; fluorescent labels such as fluorescein and its derivatives, rhodamine and its derivatives, dansyl and umbelliferone; chemiluminescent labels such as the various luciferin compounds; and enzymes such as horseradish peroxidase, alkaline phosphatase, lysozyme and glucose-6-phosphate dehydrogenase.

The antibody or reporter gene product, as the case may be, can be tagged with such labels by known methods. For example, coupling agents such as aldehydes, carbodiimides, dimaleimide, imidates, succinimides, bisdiazotized benzadine and the like may be used to tag the antibodies with fluorescent, chemiluminescent or enzyme labels.

In competitive immunoassays, samples from induced cultures (following cell disruption if the reporter gene product is not secreted) are incubated with an antibody against the reporter gene product and a known amount of labeled reporter gene product. Any unlabeled product produced by the cells competes with the labeled material for binding to the antibody. The resulting immune complexes are separated and the amount of labeled complex is determined. The reporter gene product produced by the cells can be quantified by comparing observed measurements to results obtained from standard curves. Direct immunoassays involve incubating culture samples with a labeled antibody against the reporter gene product and separating any immune complexes that form. The amount of label in the complexes is determined and can be quantified by comparison to standard curves.

Enzyme-linked immunosorbant assays (ELISAs) can also be carried out by well-known methods, e.g., as described in U.S. Pat. No. 4,665,018.

In screening for therapeutic agents for osteoporosis, cells are provided which are transformed with one of the recombinant vectors of the invention. The cells are plated in a number of culture dishes or in multi-well culture plates in a culture medium appropriate to the kind of cells used and then contacted with samples suspected to contain therapeutic agents for osteoporosis. These samples can be, e.g., aqueous or water-miscible solutions in which isolated compounds have been dissolved, or individual or pooled fractions from purification steps such as chromatography or preparative electrophoresis. Negative (sample buffer only) and positive (known amounts of estrogen or estrogen agonist) controls are run in parallel.

The present invention provides an efficient way to screen large numbers of test compounds for those which have desirable properties for either the treatment or the prevention of various cancers (e.g. breast cancer, ovarian cancer, endometrial cancer) and other diseases (e.g. endometriosis) mediated by estrogen. The invention thus provides methods of screening for novel types of antiestrogen compounds that block the indirect estrogen response and/or block estrogen action at classical estrogen response elements. As used herein an antiestrogen is a compound that substantially inhibits estrogen activity as measured in a standard assay for estrogenic activity, for example, cellular assays as described in Webb et al. Mol. Endocrinol., 6:157-167 (1993).

After incubation of the cells for an induction period, the level of expression of the reporter gene produced by each sample is measured by an assay appropriate for the gene used. The optimal time for making the measurement is determined by routine experimentation but will typically be in the range of about 24 to 72 hours. Therapeutic agents for osteoporosis in a sample will be identified by measuring a level of reporter gene expression that is higher than the unstimulated (buffer control) level.

When testing an environmental compound for estrogenic activity, the methods typically comprise cultured cells that produce high levels of the human estrogen receptor. Such cells include MCF-7 cells (ATCC No. HTB 22), MDA453 cells (ATCC No. HTB 131), ZR-75-1 cells (ATCC No. CRL 1500) or ERC1 cells described in Kushner et al., Mol. Endocrinol., 4:1465-1473 (1990). ERC2 and ERC3 cells as described by Webb, et al. Mol. Endocrinol., 6:157-167 (1993).

Cells expressing mutant estrogen receptors with decreased sensitivity for estrogenic compounds are may be used for testing environmental compounds. Cells expressing the wild type receptor (e.g., MCF7 cells) may be also used. In another embodiment, cells for the screening assay may include cells, which over-express mutant estrogen receptors, such as the ERC cells noted above.

In addition, these cells may be transfected with reporter genes in which other response element (for example the AP1) regulates expression of a reporter gene. Typically, two different reporter genes are used. One gene reports transcription induced by the estrogen response system of the invention, while the other gene reports transcription induced by the indirect estrogen response. The two reporter genes and response elements are typically placed in separate cells, but the methods can also be used with both constructs in the same cell.

DNA regions are operably linked when they are functionally related to each other. For example, a promoter is operably linked to a coding sequence if it controls the transcription of the sequence; a ribosome-binding site is operably linked to a coding sequence if it is positioned so as to permit translation. Generally, operably linked means contiguous.

Cultures of cells derived from multicellular organisms are desirable hosts for expression of the estrogen responsive element of the invention. In principle, any higher eukaryotic cell culture that either naturally expresses the estrogen receptor, or that has been genetically modified to express the estrogen receptor [(or part of that receptor)] is useable. Mammalian cells are preferred, as illustrated in the Examples. Propagation of such cells in cell culture has become a routine procedure. See Tissue Culture, Academic Press, Kruse & Patterson, editors (1973). Examples of useful host cell lines are MCF-7, MG63, HeLa, RL95.2, HepG2 and CHO cells (all available from the American Type Culture Collection, Rockville, Md.). For the purposes of the present invention, use of the MCF-7 cell line is particularly preferred, as this cell line constitutively expresses estrogen receptor.

In summary, the examples of the invention demonstrate that E2 regulation of mouse BMP-2 gene transcription requires a variant ERE binding site in the BMP-2 promoter, and that ER alpha is the dominant activator of gene expression. These findings provide a mechanistic explanation for the effects of estrogens in the pathophysiology of osteoporosis and the anabolic effects of high doses of estrogens on the skeleton.

In another embodiment, this invention provides a method of regulating expression of BMP-2 in a subject comprising the steps of administering a vector comprising an isolated nucleic acid corresponding to BMP-2 regulatory region, or a fragment thereof that comprises an estrogen responsive element and is operably linked to a nucleic acid which encodes the BMP-2 protein; and administering to the subject an effective amount of estrogen or estrogen agonist; thereby regulating expression of BMP-2 in the subject.

In another embodiment, the invention is related to the field of gynecology and fertility. The estrogen responsive element may be used to regulate expression of genes such as hormones, for example without being limited, of LH or FSH.

In another embodiment, this invention provides a method of regulating expression of BMP-2 in a subject comprising the steps of: administering to the subject an effective amount of a cell comprising the BMP-2 regulatory region, or a fragment thereof that comprises an estrogen responsive element; and administering to the subject in need an effective amount of estrogen or estrogen agonist; thereby regulating expression of BMP-2 in the subject.

In another embodiment, this invention provides a method of increasing responsiveness of a cell to estrogen or estrogen agonist comprising the step of administering a vector comprising an isolated nucleic acid corresponding to BMP-2 regulatory region, or a fragment thereof that comprises an estrogen responsive element and is operably linked to a second nucleic acid; thereby increasing the responsiveness of the cell to estrogen.

The cell can be a cell in the subject, from a subject or in another embodiment any such cell which include, but are not limited to, yeast cells, plant cells, fungal cells, insect cells, e.g. Schneider and sF9 cells, mammalian cells, e.g. HeLa cells (human), NIH3T3 (murine), RK13 (rabbit) cells, embryonic stem cell lines, e.g., D3 and J1, and cell types such as hematopoietic stem cells, myoblasts, hepatocytes, lymphocytes, airway epithelium and skin epithelium or Recombinant Eukaryotic Host.

The modified cell can be than implanted in a subject in need so as to induce the responsiveness of certain genes to estrogen or to agonist thereof in the subject in need.

In another embodiment, the invention provides a method of inhibiting response, or an oversensitive response of certain genes, to estrogen or agonist thereof by repressing the estrogen responsive element of the invention. This could be done by using the affinity to the estrogen responsive element of the invention as a decoy for the binding of Ers thereby by introducing to a cell large amount of the decoy to inhibit binding of ER to the functional EREs on the genome.

In another embodiment, this invention provides a method of enhancing repair of a bone in the body in a subject in need comprising the steps of: administering a vector comprising an isolated nucleic acid corresponding to BMP-2 regulatory region, or a fragment thereof that comprises an estrogen responsive element and is operably linked to a second nucleic acid; and administering to the subject in need an effective amount of estrogen or estrogen agonist; thereby enhancing repair of the bone in the body of the subject in need.

In another embodiment, this invention provides a method of enhancing repair of a bone comprising the steps of: administering to a subject an effective amount of a cell comprising an isolated nucleic acid corresponding to BMP-2 regulatory region, or a fragment thereof that comprises an estrogen responsive element and is operably linked to a second nucleic acid; and administering to the subject in need an effective amount of estrogen or estrogen agonist; thereby enhancing repair of the bone in the subject.

In another embodiment this invention provides a method for maintaining or increasing bone volume, bone quality, or bone strength in a subject in need afflicted with osteoporosis caused by or accompanied by a decrease in estrogen comprising the steps of: administering a vector an isolated nucleic acid corresponding to BMP-2 regulatory region, or a fragment thereof that comprises an estrogen responsive element and is operably linked to a second nucleic acid; and administering to the subject in need an effective amount of estrogen or estrogen agonist; thereby maintaining or increasing bone volume, bone quality, or bone strength in the subject in need.

In another embodiment, this invention provides a method for maintaining or increasing bone volume, bone quality, or bone strength in a subject in need afflicted with osteoporosis caused by or accompanied by a decrease in estrogen comprising the steps of: administering to a subject an effective amount of a cell comprising an isolated nucleic acid corresponding to BMP-2 regulatory region, or a fragment thereof that comprises an estrogen responsive element and is operably linked to a second nucleic acid; and administering to the subject in need an effective amount of estrogen or estrogen agonist; thereby maintaining or increasing bone volume, bone quality, or bone strength in the subject in need.

In another emodiment, this invention provides a method of enhancing repair of a bone in the body in a subject in need comprising the steps of: obtaining a cell from of the subject; transfecting the cell with a vector comprising an isolated nucleic acid corresponding to BMP-2 regulatory region, or a fragment thereof that comprises an estrogen responsive element and is operably linked to a second nucleic acid, administering the engineered cell to the subject; and administering to the subject in need an effective amount of estrogen or estrogen agonist; thereby enhancing repair of a bone in the body in the subject in need.

In another embodiment, this invention provides a method for maintaining or increasing bone volume, bone quality, or bone strength in a subject in need afflicted with osteoporosis caused by or accompanied by a decrease in estrogen comprising the steps of: obtaining a cell from of the subject; transfecting the cell with a vector comprising an isolated nucleic acid corresponding to BMP-2 regulatory region, or a fragment thereof that comprises an estrogen responsive element and is operably linked to a second nucleic acid; administering the engineered cell to the subject; and administering to the subject in need an effective amount of estrogen or estrogen agonist; thereby maintaining or increasing bone volume, bone quality, or bone strength in a subject in need.

In another embodiment, this invention provides a method for the production of transplantable bone matrix, the method comprising the steps of: obtaining a cell; transfecting the cell with a vector comprising an isolated nucleic acid corresponding to BMP-2 regulatory region, or a fragment thereof that comprises an estrogen responsive element and is operably linked to a second nucleic acid; and culturing the cell with the cell-associated matrix for a time effective for allowing formation of a transplantable bone matrix.

In another embodiment, this invention provides a method of stimulating osteoblast differentiation comprising the steps of: administering a vector an isolated nucleic acid corresponding to BMP-2 regulatory region, or a fragment thereof that comprises an estrogen responsive element and is operably linked to a second nucleic acid; and administering an effective amount of estrogen or estrogen agonist; thereby regulating expression of stimulating osteoblast differentiation.

In another embodiment, this invention provides a method of treating a bone disease in a subject comprising the steps of: administering a vector an isolated nucleic acid corresponding to BMP-2 regulatory region, or a fragment thereof that comprises an estrogen responsive element and is operably linked to a second nucleic acid and administering to the subject an effective amount of estrogen or estrogen agonist; thereby treating a bone disease in the subject.

In another embodiment, this invention provides a method of treating a bone disease in a subject comprising the steps of: administering to the subject an effective amount of a cell comprising an isolated nucleic acid corresponding to BMP-2 regulatory region, or a fragment thereof that comprises an estrogen responsive element and is operably linked to a second nucleic acid; and administering to the subject in need an effective amount of estrogen or estrogen agonist; thereby treating a bone disease in the subject.

SERMs like tamoxifen and raloxifene are therapeutic agents for several indications including the treatment and/or prevention of breast cancer and osteoporosis, and they also have potentially beneficial estrogen-like effects on the cardiovascular system (Paech et al. 1997; Black et al. 1994; Sato et al. 1996; Yang et al. 1996a; Yang et al. 1996b). Recently, raloxifene was approved for prevention and treatment of osteoporosis (Clemett and Spencer 2000). This SERM is less potent than many steroidal estrogens at maintaining bone mineral density (Sato et al. 1996) and does not improve cognitive function (Nickelsen et al. 1999) or prevent hip fractures (Ettinger et al. 1999). Thus, the quest for superior SERMs for hormone replacement therapy (HRT) continues to be an intense area of research (An et al. 2001). As demonstrated herein, the results show that SERMs such as tamoxifen and raloxifene are weak activators of the mouse BMP-2 promoter via ERα, but not ERβ. These SERMS have similar effects on the stimulation of human BMP4 promoter activity. Phytoestrogens such as genistein exhibit some preference for ERβ versus ERα (An et al. 2001). Consistent with its moderate binding selectivity, it was shown in the present invention that genistein triggers the transcriptional activation pathways of the mouse BMP-2 gene with ERβ, but not with ERα. The invention herein is widely applicable to a variety of situations where it is desirable to be able to turn gene expression “on” and “off”, or regulate the level of gene expression, in a rapid, efficient and controlled manner without causing pleiotropic effects or cytotoxicity. The invention is useful for gene therapy purposes in humans, in treatments for either genetic or acquired diseases. The general approach of gene therapy involves the introduction of one or more nucleic acid molecules into cells such that one or more gene products encoded by the introduced genetic material are produced in the cells to restore or enhance a functional activity. However current gene therapy vectors typically utilize constitutive regulatory elements which are responsive to endogenous transcriptions factors. These vector systems do not allow for the ability to modulate the level of gene expression in a subject. In contrast, the regulatory system of the invention provides this ability.

In one embodiment, the cell or the vector of the invention system may comprise a promoter which is tissue or organ specific (for example, brain, heart or blood vessel) so to enable the expression of the genes in the specific organs or tissue. In another embodiment, the may be applied to the specific tissue or organ by using delivery methods which are well known in the art. Thus, the regulatory system of the invention offers the advantage over constitutive regulatory systems of allowing for modulation of the level of gene expression depending upon the requirements of the therapeutic situation.

The regulatory system of the invention can also be used to express a suicide gene (such as a ricin or HSV tk gene) in cells in a conditional manner to allow for destruction of the cells (e.g., in vivo) following a particular therapy. For example, a suicide gene can be introduced into tumor cells to be used for anti-cancer immunization or into the viral genome of a live attenuated viral to be used as a vaccine. The tumor cells or viral vaccine carrying the suicide gene are administered to a subject in the presence of Tc (or analogue thereof). Following immunization, the drug is withdrawn (e.g., administration is stopped), thereby inducing expression of the suicide gene to destroy the tumor cells or cells carrying the live virus.

Cells types, which can be modified for gene therapy purposes include hematopoietic stem cells, myoblasts, hepatocytes, lymphocytes, airway epithelium and skin epithelium. For further descriptions of cell types, genes and methods for gene therapy see e.g., Wilson, J. M et al. (1988) Proc. Natl. Acad. Sci. USA 85:3014-3018; Armentano, D. et al. (1990) Proc. Natl. Acad. Sci. USA 87:6141-6145; Wolff, J. A. et al. (1990) Science 247:1465-1468; Chowdhury, J. R. et al. (1991) Science 254:1802-1805; Ferry, N. et al. (1991) Proc. Natl. Acad. Sci. USA 88:8377-8381; Wilson, J. M. et al. (1992) J. Biol. Chem. 267:963-967; Quantin, B. et al. (1992) Proc. Natl. Acad. Sci. USA 89:2581-2584; Dai, Y. et al. (1992) Proc. Natl. Acad. Sci. USA 89:10892-10895; van Beusechem, V. W. et al. (1992) Proc. Natl. Acad. Sci. USA 89:7640-7644; Rosenfeld, M. A. et al. (1992) Cell 68:143-155; Kay, M. A. et al. (1992) Human Gene Therapy 3:641-647; Cristiano, R. J. et al. (1993) Proc. Natl. Acad. Sci. USA 90:2122-2126; Hwu, P. et al. (1993) J. Immunol. 150:4104-4115; and Herz, J. and Gerard, R. D. (1993) Proc. Natl. Acad. Sci. USA 90:2812-2816.

The regulatory system of the invention can also be used to produce and isolate a gene product (e.g., protein) of interest. Large scale production of a protein of interest can be accomplished using cultured cells in vitro which have been modified to contain 1) nucleic acid encoding a estrogen responsive element of the invention and 2) a second nucleic acid (e.g., encoding a protein of interest) operatively linked to a BMP-2 promoter or fragment thereof which contain an estrogen responsive element of the invention. For example, mammalian, yeast or fungal cells can be modified to contain these nucleic acid components as described herein. Alternatively, an insect cell/baculovirus expression system can be used. To produce and isolate a gene product of interest, a host cell (e.g., mammalian, yeast or fungal cell) BMP-2 promoter or fragment thereof which contain an estrogen responsive element of the invention and a second nucleic acid linked to nucleic acid encoding the gene product of interest, are first grown in a culture medium in the absence of estrogen. Under these conditions, expression of the second nucleic acid is repressed. Next, the concentration of estrogen or estrogen analogue in the culture medium is increased to stimulate transcription of the a second nucleic acid. The gene product can then be isolated from harvested cells or from the culture medium by standard techniques.

The invention also provides for large-scale production of a protein of interest in animals, such as in transgenic farm animals. Advances in transgenic technology have made it possible to produce transgenic livestock, such as cattle, goats, pigs and sheep (reviewed in Wall, R. J. et al. (1992) J. Cell. Biochem. 49:113-120; and Clark, A. J. et al. (1987) Trends in Biotechnology 5:20-24). Accordingly, transgenic livestock carrying in their genome the components of the regulatory system of the invention can be constructed.

A transgenic animal can be created, for example, by introducing a nucleic acid encoding a protein of interest linked estrogen regulatory elements of the invention, into the male pronuclei of a fertilized oocyte, e.g., by microinjection, and allowing the oocyte to develop in a pseudopregnant female foster animal. Intronic sequences and polyadenylation signals can also be included in the transgene to increase the efficiency of expression of the transgene. Methods for generating transgenic animals, particularly animals such as mice, have become conventional in the art and are described, for example, in U.S. Pat. Nos. 4,736,866 and 4,870,009 and Hogan, B. et al., (1986) A Laboratory Manual, Cold Spring Harbor, N.Y., Cold Spring Harbor Laboratory. A transgenic founder animal can be used to breed additional animals carrying the transgene. A transgenic animal carrying one transgene can further be bred to another transgenic animal carrying a second transgenes to create a so-called “double transgenic” animal carrying two transgenes.

### EXAMPLES

Materials and Methods

Chemical Reagents

All materials were purchased from Sigma Chemical Co. (St. Louis, Mo.) unless otherwise stated. DMEM, penicillin-streptomycin, L-glutamine were purchased from Biological Industries (Beit Haemek, Israel). ICI-182,780 was purchased from Zeneca Pharmaceuticals, UK.

Plasmid Construction

Express vectors for human ERα and human ERβ (485) were previously described (Webb et al. 1998). Full length (−2712 to +165) and 5′-end deletions of the mouse BMP-2 promoter (−838 to +165, and −150 to +165) were cloned upstream of the luciferase cDNA in the pGL3 vector (Promega) as previously described (Harris et al. 2000). Mutation of the mouse BMP-2 variant ERE (Δvariant ERE: 5′-GAACCActcTACCTC-3′) in the full-length promoter plasmid was accomplished using the QuikChange site-directed mutatgesis kit (Stratagene, USA) according to the manufacturer's protocol. The promoter fragments were subcloned as PCR products into the pGL3-basic vector (−448 to +23 and −400 to +23). ERE-tk-luciferase vectors (one copy of the ERE from the frog vitellogenin A2 gene) were constructed as previously described (An et al. 1999).

Animal and Cell Culture

Two months old Swiss-Webster female mice (ICR) were OVX in accordance with mandated standards of humane care, and the animals were maintained in accordance with the NIH Guide for the Care and Use of Laboratory Animals. After 5 months post-surgery, bone marrow was isolated from femurs and tibias, and the MSCs were cultured as described previously (Gazit et al. 1999 and Zhou et al. 2001). The bone marrow cells were maintained in DMEM (Phenol red free, 1.0 g/L glucose, Biological Industries, Israel) with 15% FBS (Charcoal stripped, heat-inactivated), 100 units/ml penicillin, 100 μg/ml streptomycin, and 2 mM glutamine. At day 4, the cultures were supplemented with 50 μg/ml ascorbic acid, 10 mM β-glycerophosphate, and 10 nM dexamethasone. From day 10, the cells were cultured in DMEM with 2% charcoal stripped (CS)-FBS without osteogenic supplements. At day 11, the cultures were treated with E2 (Sigma), ICI-182,780 (AstraZeneca Pharmaceuticals, UK), tamoxifen (Sigma) or raloxifene for 24 hr. RNA then was isolated at day 12. To determine whether E2 directly regulated BMP-2 mRNA expression in mouse MSCs, 5.0 μM cycloheximide was added to the cultures with fresh DMEM plus 2% CS-FBS for 45 min before 100 nM E2 treatment, and RNA was isolated 4 hr after E2 treatment. Mouse C3H10T1/2 cells were cultivated in DMEM (Sigma and Biological Industries) with 10% FBS, 100 units/ml penicillin, 100 μg/ml streptomycin, and 2 mM glutamine.

Cell Transfection and Luciferase Assays

Transient transfection was performed as previously described (An et al. 1999). Briefly, C3H10T1/2 cells were cultured in 100-mm dishes until confluent. The cells were harvested by trypsinization, resuspended in medium, counted, pelleted at 800 rpm for 5 min, and 1.5×10⁷ cells were resuspended again in 0.5 ml PBS containing 0.1% glucose. The cell suspension was mixed with 5 μg luciferase reporter plasmids and 2 μg hERα or hERβ expression vectors. The cells were transferred to a cuvette and electroporated using a Bio-Rad gene pulser. After electroporation, the cells were suspended in DMEM (phenol red free) containing 2% CS-FBS and seeded at 1 ml per well into 12-well multi-plates. The cells were treated with E2 (10-8 M) or ethanol (vehicle) for 24 hr, and luciferase activity was assayed using a kit from Promega with a luminometer (Turner Designs TD-20/20, CA). The efficiency of transfection was monitored by co-transfection of 0.5 μg of pNGVL1-nt-betaGa1 plasmids (Constructed by National Gene Vector Laboratory at the University of Michigan, Ann Arbor, USA), and β-galactosidase activity was measured using the Galacto-Light Chemiluminescent Reporter Assay System Kit (Tropix of PE Biosystems, USA). The transfection results were reported as the fold induction of RLU (Relative Light Units) for E2 treated cells over vehicle control treated cells after normalization to β-galactosidase expression. Error bars show the standard error among five experiments, each done in triplicate.

RNA Isolation, Semi-Quantitative RT-PCR and Real-Time RT-PCR

RNA was isolated using TRizol Reagent (Life Technologies, USA), according to the manufacturer's protocol. Semi-quantitative RT-PCR was performed as described previously (Zhou et al. 2001). Mouse BMP-2 (505 bp) (Zhou et al. 2001), internal control RPL19 (190 bp) (Orly et al. 1994) and c-myc (550 bp) (Goswami et al. 1997) primers were described previously. The PCR conditions used for mouse BMP-2 RT-PCR were 30 cycles of 94 C for 1 min, 55 C for 1 min and 72 for 2 min in an MJ MiniCycler (MJ Research, USA). RT-PCR products of mouse BMP-2 were cloned into the pGEM-T Easy vector (A1360, Promega), and the pGEM-T-mouse BMP-2 vectors were sequenced by a T7 sequence sequencing kit (US70770, USB, Cleveland, USA) according to the manufacturer's protocols. DNA sequence analysis confirmed that mouse BMP-2 had been amplified.

Real-Time PCR was performed using a Roche LightCycler according to the manufacturer's protocol (Roche Molecular Biochemicals, USA). After reverse transcription reaction using 2 μg of total RNA, real-time PCR was carried out in a 20 μl final volume using the LightCycler-FastStart DNA Master SYBR Green I kit (Roche). The reaction mix contained 1×LightCycler-FastStart Master SYBR Green I, 0.5 μM of each primer, 4 mM MgCl₂, and 2 μl cDNA from RT reaction. The conditions of the real-time PCR were as follows: 95° C. 10 min for one cycle to activate the modified FastStart Taq DNA polymerase, followed by 45 cycles at 95° C. for 15 s, 60° C. to 55° C. touchdown at steps of 0.5° C. for 10 s, and 72° C. for 25 s. Fluoresence was measured at 82° C. for 5 s. To quantify the copy number of the mouse BMP-2 mRNA in MSCs, pGEM-T-mouse BMP-2 plasmids (102 to 108 copies) were used in standard curve.

Statistical Analysis

All experiments were performed three to five times independently. Data re presented as the mean values±the standard error of the mean. The semi-quantitative RT-PCR and real-time RT-PCR were performed 3 times in independent experiments using total RNA that was isolated from MSCs derived from 3-6 animals each time. Quantitative data were analyzed using either the non-parameteric Mann-Whitney test or the ANOVA test.

### EXPERIMENTAL RESULTS

### Example 1

### E2 Directly Regulates BMP-2 mRNA Expression in Mouse MSCs

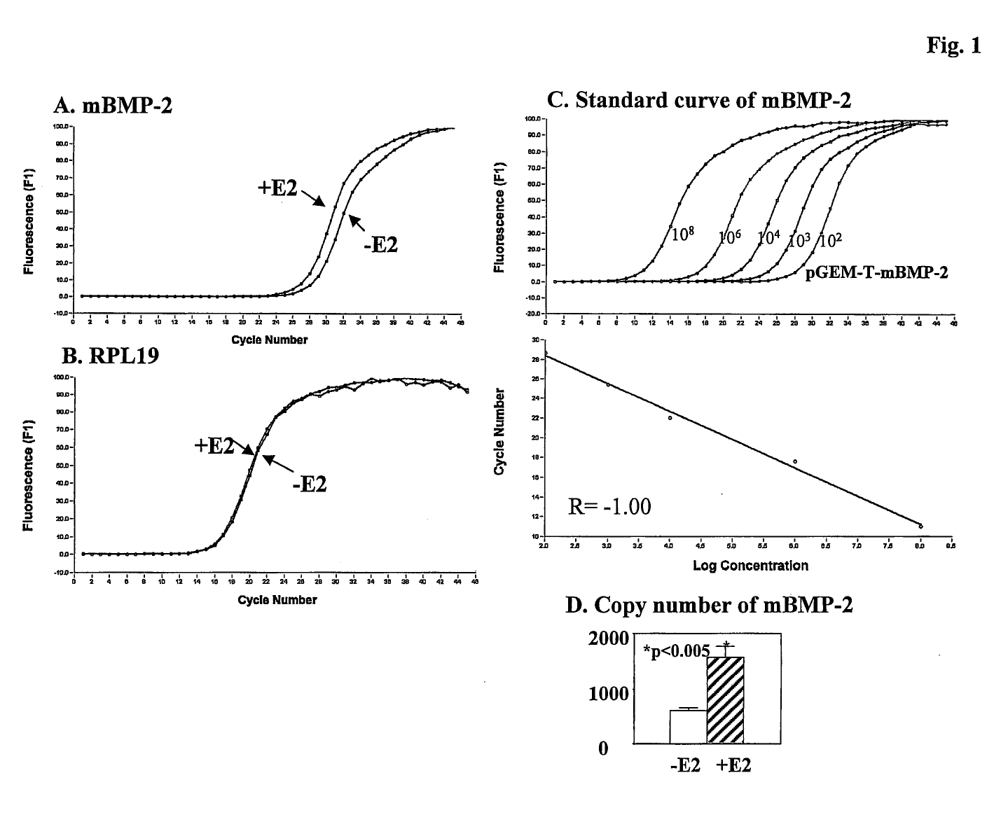

Bone marrow MSCs obtained from ovaryectomized mice (5 months after surgery) express BMP-2 mRNA s shown by real-time RT-PCR (FIG. 1A). After 24 hr of treatment with 100 nM E2, mouse BMP-2 mRNA levels were significantly increased by 2.4-fold from 570±81 copies to 1337±177 copies (p<0.05, ANOVA) in 2 ug of total RNA (FIG. 1D). The ribosomal protein L19 (RPL19) served as an internal control, and its expression was not altered by E2 treatment (FIG. 1B).

In order to exclude the possibility that the PCR primers for mouse BMP-2 were amplifying a mRNA sequence other than the intended target, the amplification products were purified, cloned and sequenced. A subsequent BLAST analysis (data not shown) identified sequences corresponding to mouse BMP-2 as listed in the GeneBank database (Feng et al. 1994; accession number NM 007553). The cloned mouse BMP-2 cDNA product (pGEM-T-mouse BMP-2 vector) was then used in real-time RT-PCR to generate the standard curve for the mouse BMP-2 gene (FIG. 1C).

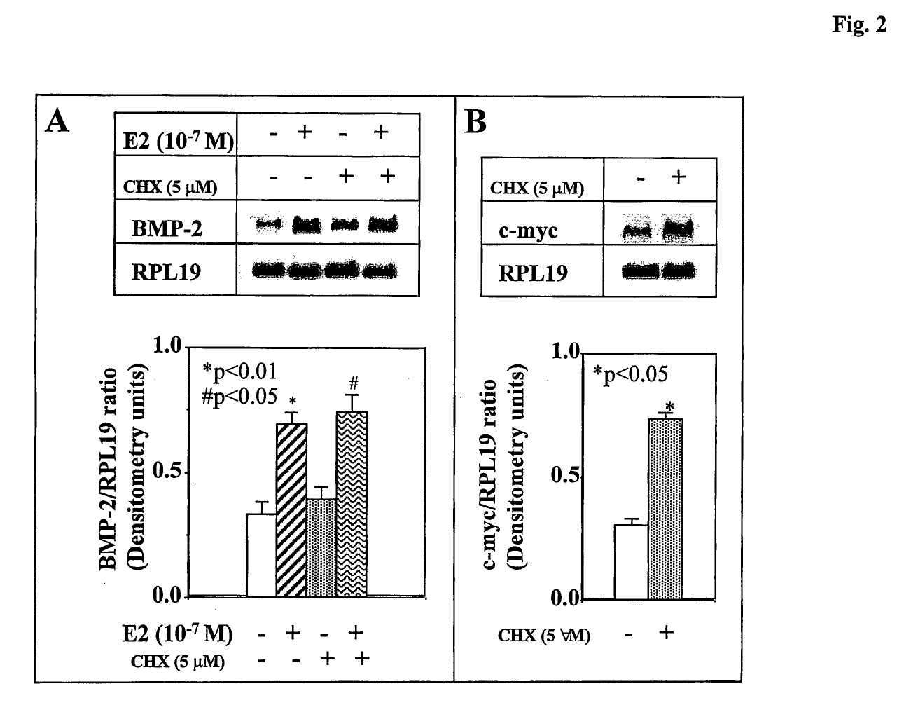

As shown in FIG. 2A, after 24 hr of treatment of mouse MSCs with 100 nM E2, there was an up-regulation of BMP-2 mRNA levels as determined by semi-quantitative RT-PCR. Co-treatment with 5.0 uM cycloheximide (an inhibitor of protein synthesis) did not block this increase in BMP-2 mRNA, although the same concentration of cycloheximide caused a super-induction of c-myc mRNA implying that it was effective at inhibiting protein synthesis (Hauguel-de Mouzon and Kahn 1991) (FIG. 2B). This result demonstrates that E2 regulation of mouse BMP-2 mRNA in MSCs is direct and independent of ongoing protein synthesis.

### Example 2

### E2 Regulation of BMP-2 mRNA Expression in Mouse MSCs is ER Dependent

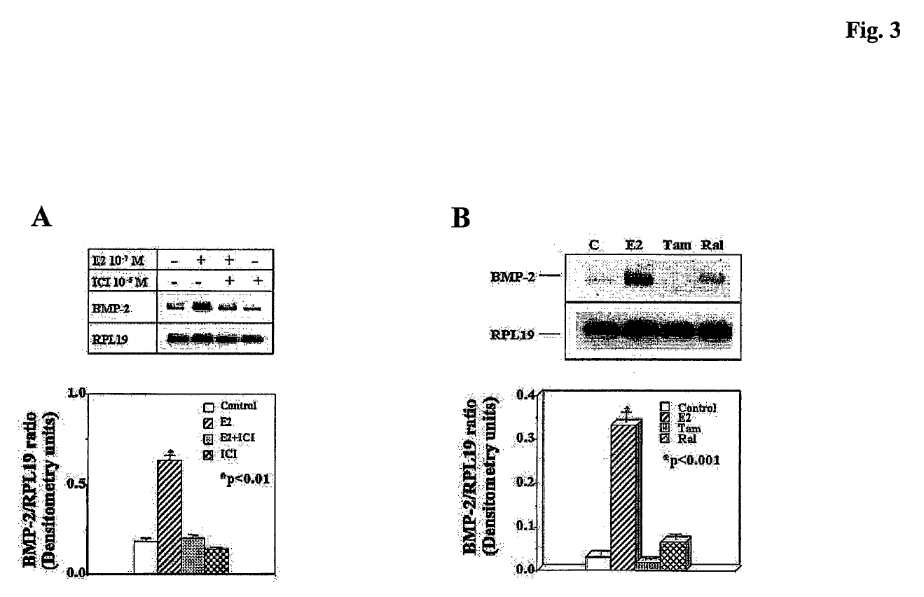

As determined by semi-quantitative RT-PCR, after a 24 hr treatment period, the ER antagonist ICI (10 μM) alone had no effect on constitutive mouse BMP-2 mRNA levels (FIG. 3A). However, it blocked the up-regulation of BMP-2 mRNA expression by E2 (100 nM) in mouse MSCs, demonstrating that E2 regulates mouse BMP-2 gene expression in MSCs via ERs. In addition, mouse BMP-2 mRNA expression was up-regulated by E2 (100 nM) treatment of MSCs, but not by selective estrogen receptor modulators such as tamoxifen (1.0 μM) or raloxifene (100 nM) (FIG. 3B).

### Example 3

### E2 Dose-Dependently Regulates Mouse BMP-2 Promoter Activity via ERα and ERβ in C3H10T1/2 Cells

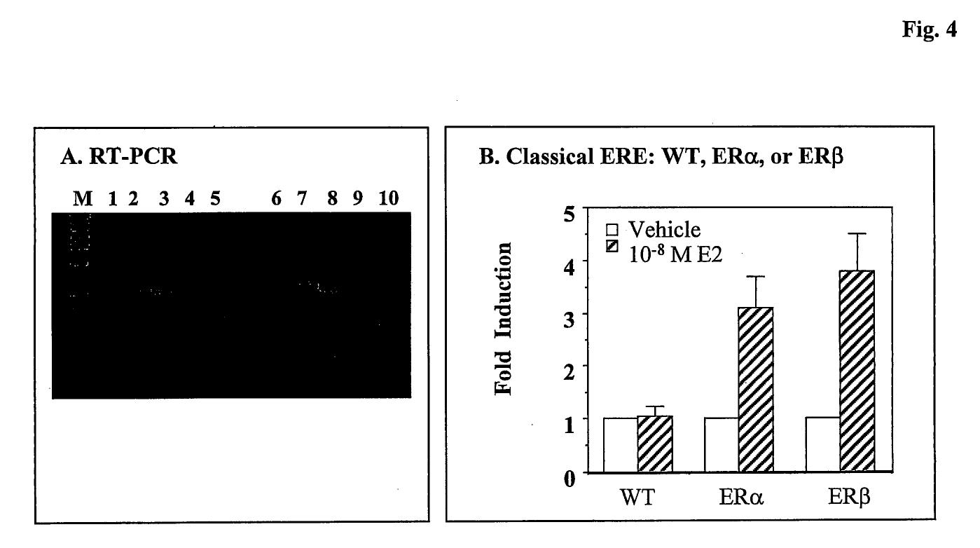

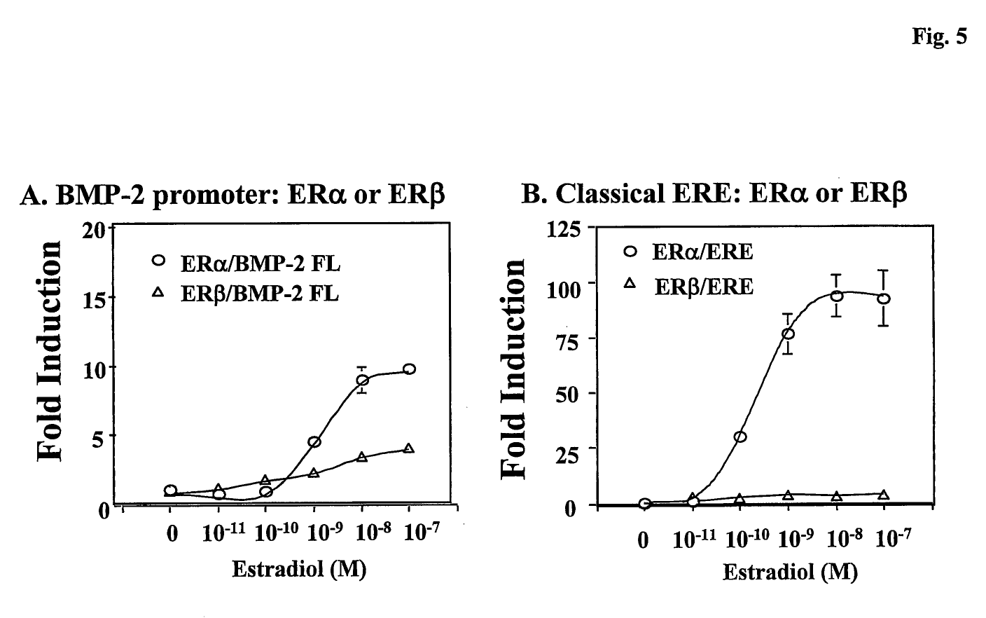

In order to test the hypothesis that estrogens transcriptionally activate mouse BMP-2 gene expression via n variant estrogen responsive element binding site, the effect of E2 on mouse BMP-2 promoter activity was examined in the mesenchymal stem cell line C3H10T1/2. This cell line was used, because mouse C3H10T1/2 cells do not express detectable levels of ERs and therefore require transfection of ERs to elicit E2 effects on transcription (FIG. 4). Full-length mouse BMP-2 promoter (−2712)-luciferase or classical ERE-tk-luciferase (An et al., 1999) plasmids were transiently co-transfected into C3H10T1/2 cells with either human ER□ or ER□ expression vectors. The cells were then treated for 24 hr with different concentrations of E2, and luciferase activity was assayed by a luminometer. The results (FIG. 5A) showed that E2 via either ERα or ERβ, up-regulated BMP-2 promoter (−2712) activity in a dose-dependent manner, although ERα was the more efficacious activator of both the mouse BMP-2 promoter and the classical ERE (FIG. 5B).

### Example 4

### E2 Stimulation of Mouse BMP-2 Promoter Activity is ER Dependent

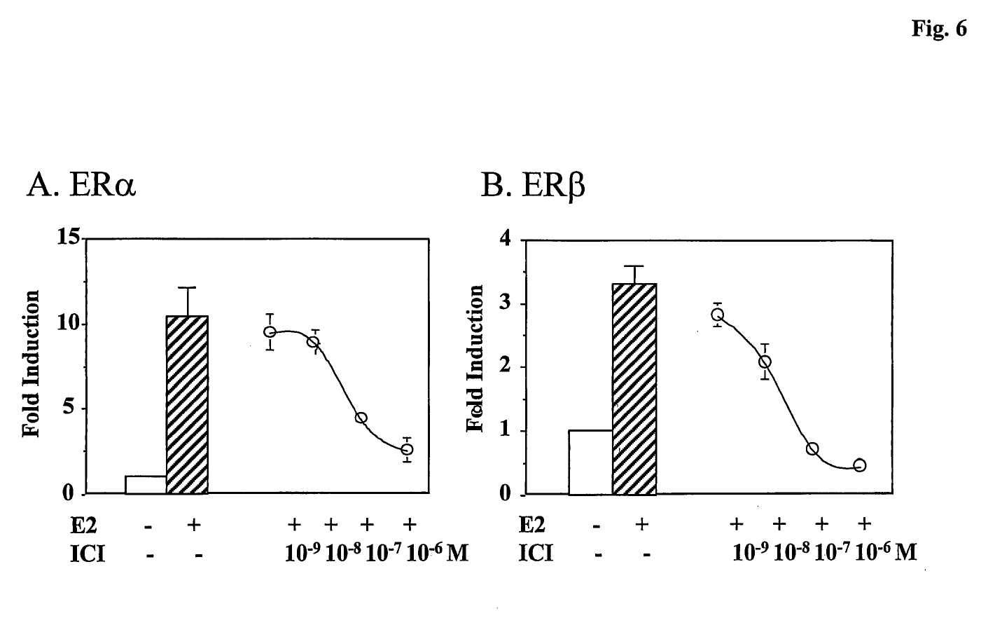

As is shown in FIG. 6, the ER antagonist ICI dose-dependently inhibited the stimulation of mouse BMP-2 promoter (−2712) activity by 10 nM E2 through either ERα or ERβ. These luciferase assay results were in agreement with the BMP-2 mRNA expression data obtained with mouse bone marrow MSCs that were co-treated with E2 and ICI (FIG. 3).

### Example 5

### Location of an ER Regulatory Site in the Mouse BMP-2 Promoter

Harris et al. (2000) had cloned and sequenced the mouse BMP-2 promoter (−2712 to +165), and reported that it contains several cis-acting DNA control elements including Sp1 and AP-1. In addition, in the present invention, a previously unrecognized variant non-palindromic ERE (5′-GGGCCActcTGACCC-3′) (SEQ ID NO: 4) that is located at −415 to −402 of the promoter, was identified. Heller et al. (1999) also cloned the mouse BMP-2 promoter (−3365 to −1658), and like Harris et al. (2000), these authors did not report the existence of an estrogen responsive element-like sequence.

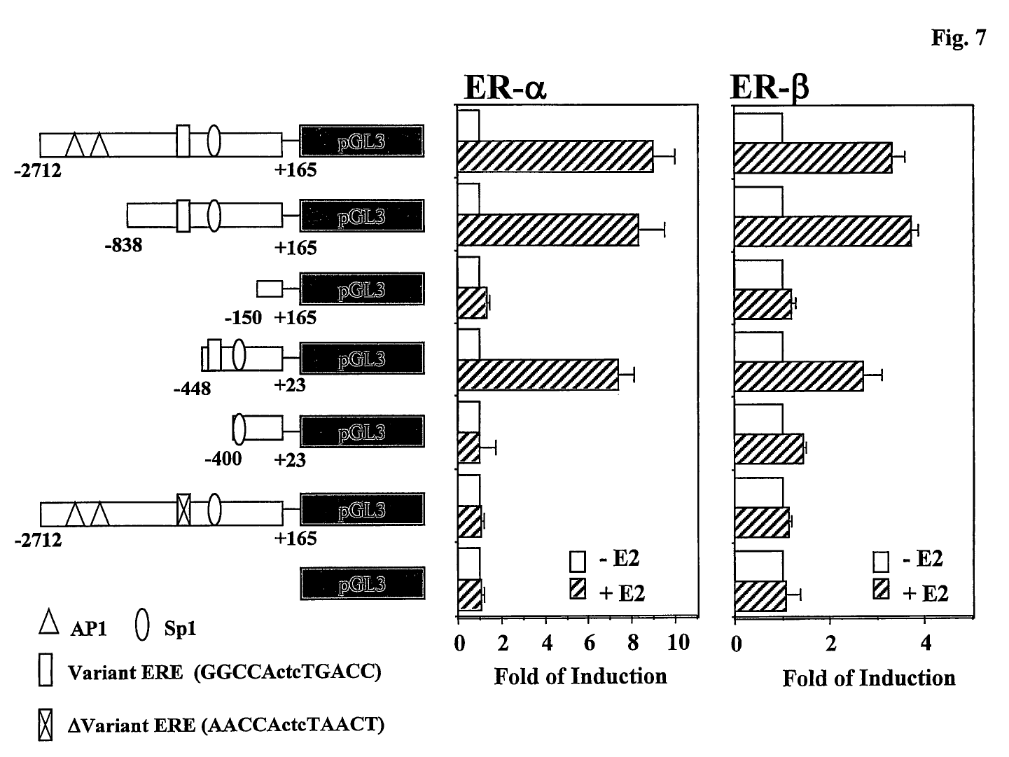

In order to find the regulatory site(s) for the ER in the mouse BMP-2 promoter, we compared the activity of the full-length promoter (−2712) to different promoter deletions as well as to mutation of the putative variant ERE (FIG. 7). The full-length promoter (−2712) contains two AP-1 response elements, one GC-rich Sp1 site and a possible variant ERE, all of which the ER could operate through (Paech et al. 1997). The −838 fragment contains the Sp1 site and the putative variant ERE but lacks the two AP-1 response elements, while the −150 fragment is without any of these sites. The −448 fragment still contains the Sp1 and variant ERE sites, while the −400 fragment lacks the variant ERE but retains the Sp1 site. Finally, the putative variant ERE was also mutated (Δvariant ERE: 5′-GAACCActcTACCTC-3′) (SEQ ID NO: 5) in the full-length promoter (−2712), while leaving the other regulatory sites intact. These different mouse BMP-2 promoter-luciferase constructs were transiently co-transfected with either human ERα or ERβ expression vectors into C3H10T1/2 cells, and luciferase activity was assayed after 24 hr of treatment with 10 nM E2.

As shown in FIG. 7, E2 acting through either ERα or ERβ up-regulated activity of the full-length (−2712) as well as the −838 and −448 fragments of the mouse BMP-2 promoter, but did not increase expression of the −150 fragment which lacks all of these regulatory sites. Since there was no difference between the activities of the full-length (−2712) and the −838 and −448 fragments, the AP-1 response elements were not required for E2 induction. On the other hand, deletion (−400) or mutation (Δvariant ERE) of the putative variant ERE eliminated the ability of E2 to increase mouse BMP-2 promoter activity via either ERα or ERβ. Thus, the Sp1 site does not appear to be important for ER action on the promoter, while the putative variant ERE seems to be critical for the hormone's effect.

### Example 6

### Stimulation of the Mouse BMP-2 Promoter by Selective Estrogen Receptor Modulator and Genistein

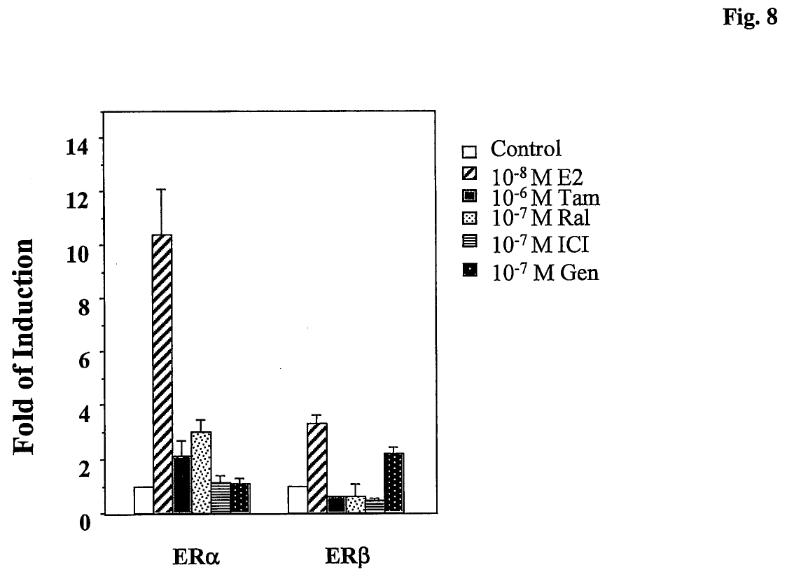

In order to test whether selective estrogen receptor modulators and genistein as well as E2 regulate mouse BMP-2 promoter activity, the full-length promoter (−2712)-luciferase plasmid was transiently co-transfected into C3H10T1/2 cells with either human ERα or ERβ. After transfection, the cells were treated with either vehicle (ethanol control), 10 nM E2, 100 nM raloxifene, 1.0 μM tamoxifen, 100 nM genistein or 100 nM ICI for 24 hr, and luciferase activity was then assayed by a luminometer. As shown in FIG. 8, tamoxifen and raloxifene are partial agonists of the BMP-2 promoter via ERα, but not via ERβ.

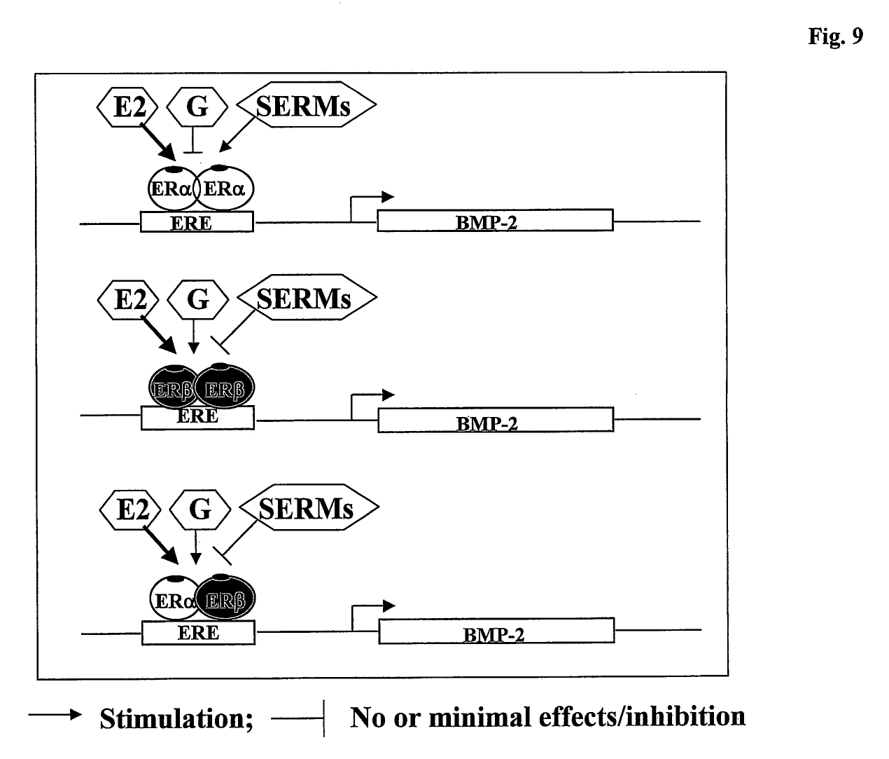

As also shown in FIG. 8, genistein stimulates mouse BMP-2 promoter activity too, but this effect is mediated via ERα, and not via ERβ. Finally, as with E2 action, mutation of the variant ERE in the full-length (−2712) promoter abolished the stimulation of both SERMs and genistein demonstrating that the variant ERE is responsible for these effects. A summary of the above results is shown in FIG. 9.

1. An isolated nucleic acid molecule comprising a nucleic acid, which corresponds to BMP-2 regulatory region, comprising an estrogen responsive element.

2. A vector comprising the nucleic acid according to claim 1, wherein the nucleic acid is operably linked to a second nucleic acid.

3. A host cell comprising the vector of claim 2.

4. The host cell of claim 3, wherein the cell further comprises an estrogen receptor.

5. The host cell of claim 4, wherein the estrogen receptor is α.

6. The host cell of claim 4, wherein the estrogen receptor is β.

7. A method for the identification of a therapeutic agent for the prevention and/or treatment of osteoporosis, comprising:
(a) introducing into a cell the vector of claim 2;
(b) contacting the cell with a candidate agent; and
(c) monitoring the expression of the protein encoded by the reporter nucleic acid, wherein induced expression of the protein indicates that the candidate agent is a potential therapeutic agent.

8. The method of claim 7, wherein in step (a) a second expression vector comprising a nucleic acid molecule encoding an estrogen receptor is introduced into the cell.

9. The method of claim 8, wherein the estrogen receptor is α.

10. The method of claim 8, wherein the estrogen receptor is β.

11. A method of regulating expression of BMP-2 in a subject comprising the steps of: administering the vector of claim 2, wherein said second nucleic acid encodes for the BMP-2; and administering to the subject an effective amount of estrogen or estrogen agonist; thereby regulating expression of BMP-2 in the subject.

12. A method of regulating expression of BMP-2 in a subject comprising the steps of: administering to the subject an effective amount of the cell of claim 3, wherein the a second nucleic acid encodes for BMP-2; and administering to the subject in need an effective amount of estrogen or estrogen agonist; thereby regulating expression of BMP-2 in the subject.

13. The method of claim 12 wherein the cell is a mesenchymal stem cell, a progenitor cell, or a cell capable of differentiating into an osteoblast cell.

14. A method of increasing responsiveness of a cell to an estrogen or estrogen agonist comprising the step of administering the vector of claim 2; thereby increasing the responsiveness of the cell to estrogen.

15. The method of claim 14, wherein the cell comprises an estrogen receptor.

16. The method of claim 14, wherein the cell is a mesenchymal stem cell, a progenitor cell, or a cell capable of differentiating into an osteoblast cell.

17. A method of enhancing repair of a bone in the body in a subject in need comprising the steps of: administering the vector of claim 2; and administering to the subject in need an effective amount of estrogen or estrogen agonist; thereby enhancing repair of the bone in the body of the subject in need.

18. A method of enhancing repair of a bone comprising the steps of: administering to a subject an effective amount of the cell of claim 3; and administering to the subject in need an effective amount of estrogen or estrogen agonist; thereby enhancing repair of the bone in the subject.

19. The method of claim 18, wherein the cell is a mesenchymal stem cell, a progenitor cell, or a cell capable of differentiating into a osteoblast cell.

20. A method for maintaining or increasing bone volume, bone quality, or bone strength in a subject in need afflicted with osteoporosis caused by or accompanied by a decrease in estrogen comprising the steps of: administering the vector of claim 2; and administering to the subject in need an effective amount of estrogen or estrogen agonist; thereby maintaining or increasing bone volume, bone quality, or bone strength in the subject in need.

21. A method for maintaining or increasing bone volume, bone quality, or bone strength in a subject in need afflicted with osteoporosis caused by or accompanied by a decrease in estrogen comprising the steps of: administering to a subject an effective amount of the cell of claim 3; and administering to the subject in need an effective amount of estrogen or estrogen agonist; thereby maintaining or increasing bone volume, bone quality, or bone strength in the subject in need.

22. The method of claim 21, wherein the cell is a mesenchymal stem cell, a progenitor cell, or a cell capable of differentiating into a osteoblast cell.

23. A method of enhancing repair of a bone in the body in a subject in need comprising the steps of: obtaining a cell from of the subject; transfecting the cell with the vector of claim 2; administering the engineered cell to the subject; and administering to the subject in need an effective amount of estrogen or estrogen agonist; thereby enhancing repair of a bone in the body in the subject in need.

24. A method for maintaining or increasing bone volume, bone quality, or bone strength in a subject in need afflicted with osteoporosis caused by or accompanied by a decrease in estrogen comprising the steps of: obtaining a cell from of the subject; transfecting the cell with the vector of claim 2; administering the engineered cell to the subject; and administering to the subject in need an effective amount of estrogen or estrogen agonist; thereby maintaining or increasing bone volume, bone quality, or bone strength in a subject in need.

25. The method of claim 24, wherein the step of administering to the subject in need an effective amount of estrogen or estrogen agonist further increase the level of expressed BMP 2 by 1.5-30 fold.

26. The method of claim 24, wherein the cell is a mesenchymal stem cell, a progenitor cell, or a cell capable of differentiating into an osteoblast cell.

27. A method for the production of transplantable bone matrix, the method comprising the steps of: obtaining a cell; transfecting the cell with the vector of claim 2; and culturing the cell with the cell-associated matrix for a time effective for allowing formation of a transplantable bone matrix.

28. The method of claim 27, wherein the cell is a mesenchymal stem cell, a progenitor cell, or a cell capable of differentiating into an osteoblast cell.

29. A method of stimulating osteoblast differentiation comprising the steps of: administering the vector of claim 2; and administering an effective amount of estrogen or estrogen agonist; thereby regulating expression of stimulating osteoblast differentiation.

30. A method of treating a bone disease in a subject comprising the steps of: administering the vector of claim 2; and administering to the subject an effective amount of estrogen or estrogen agonist; thereby treating a bone disease in the subject.

31. A method of treating a bone disease in a subject comprising the steps of: administering to the subject an effective amount of the cell of claim 3; and administering to the subject in need an effective amount of estrogen or estrogen agonist; thereby treating a bone disease in the subject.

32. The method of claim 31, wherein the cell is a mesenchymal stem cell, a progenitor cell, or a cell capable of differentiating into an osteoblast cell.

33. A method for identifying a compound in a sample as an estrogenic agonist comprising:
(a) providing a cell line expressing receptors for human estrogen, which cell line has been stably transfected by a vector comprising a reporter nucleic acid operatively linked to an isolated nucleic acid corresponding to BMP-2 regulatory region, or a fragment thereof that comprises an estrogen responsive element, wherein said estorgen responsive element is capable of controlling expression of the reporter nucleic acid in response to estrogen;
(b) contacting the transfected cell line with a sample suspected to contain a human estrogen agonist, under conditions in which human estrogen would cause increased expression of the reporter nucleic acid; and
(c) measuring the level of expression of the reporter nucleic acid,
Whereby a human estrogen agonist in the sample is identified by measurement of an increased level of expression of the reporter nucleic acid, compared to the level produced by a buffer control.

34. A method for identifying a compound in a sample as a human estrogen antagonist comprising:
(a) providing a cell line expressing receptors for human estrogen, which cell line has been stably transfected by a vector comprising a reporter nucleic acid operatively linked to an isolated nucleic acid corresponding to BMP-2 regulatory region, or a fragment thereof that comprises an estrogen responsive element wherein said estorgen responsive element is capable of controlling expression of the reporter nucleic acid in response to estrogen;
(b) contacting the transfected cell line with a sample suspected to contain a human estrogen antagonist, to which has been added an amount of human estrogen that, absent such antagonist, would produce a measurable increase in expression of the reporter nucleic acid; and
(c) measuring the level of expression of the reporter nucleic acid,
whereby a human estrogen antagonist in the sample is identified by measurement of a decreased level of expression of the reporter nucleic acid, compared to the level produced by the human estrogen in the absence of such antagonist.
